(19) United States
(12) Patent Application Publication
(10) Pub. No.: US 2023/0084089 A1
(43) Pub. Date: Mar. 16, 2023

(54) WEARABLE MEDICAL DEVICE

(71) Applicant: West Affum Holdings Corp., Grand Cayman, KY (US)

(72) Inventor: Jaeho Kim, Redmond, WA (US)

(21) Appl. No.: 17/945,966

(22) Filed: Sep. 15, 2022

Related U.S. Application Data
(63) Continuation of application No. 16/985,017, filed on Aug. 4, 2020.
(60) Provisional application No. 62/883,784, filed on Aug. 7, 2019.

(51) Int. Cl. A61B 5/00; A61N 1/362; A61N 1/04; A61N 1/39; A61B 5/282

(52) U.S. Cl. CPC A61B 5/6804; A61N 1/3625; A61N 1/046; A61N 1/0484; A61N 1/3981; A61N 1/3993; A61N 1/3904; A61B 5/282; A61B 2562/14

(57) ABSTRACT

Technologies and implementations for a wearable medical device (WMD). The technologies and implementations facilitate improved comfort and usability of WMDs. Additionally, the technologies and implementations include WMDs having wearable cardioverter defibrillator capabilities.

### RELATED APPLICATION

This application claims benefit of priority to U.S. Provisional Patent Application Ser. No. 62/883,784, filed on Aug. 7, 2019, titled WEARABLE SUPPORT STRUCTURE INCLUDING THE ELECTRONICS MODULE FOR WEARABLE CARDIOVERTER DEFIBRILLATOR (WCD), which is incorporated herein by reference in its entirety for all purposes.

### INFORMATION

Technology has contributed to improvements in healthcare. Some examples include healthcare related devices that may be mobile and personal. Mobile and personal healthcare devices may include Wearable Medical Devices (WMDs). Some WMDs may include medical devices that facilitate monitoring of various health related activities of a person. For example, a WMD may include a medical device that may be used to monitor a person's heart activity. The heart activity monitored by the WMD may be in the form of electrical signals (i.e., electrocardiogram or ECG). The WMD may be in a form factor capable of being worn by a person, who's heart activity is to be monitored. Monitoring of a person's ECG may facilitate intervention of heart related issues.

An example of a WMD, which may be used to monitor and facilitate intervention of a person's heart activity, may be a cardioverter defibrillator type medical device (e.g., wearable cardioverter defibrillator or WCD). Some examples of WCDs may be included in a clothing having electrodes configured to facilitate providing an electrical shock to the person. Various electrical activities of the WCD may be performed by an electronic module (e.g., monitoring of the user's health, indication signals, display, power supply, electrical signal to the electrodes, user interface, etc.). Commonly the electronic module may be separate from the clothing (e.g., an electronic module configured to be carried separately from the clothing). Because the electronic module may be separately carried by the person from the clothing, the comfort and convenience of the WCD may be a negative experience for the user. For example, having the electronic module separate from the clothing, the user may carry the electronic module in a separate bag (e.g., carry bag). Carrying a separate bag having the electronic module may be inconvenient, cumbersome, and/or uncomfortable for the user.

The foregoing summary is illustrative only and not intended to be in any way limiting. In addition to the illustrative aspects, embodiments, and features described above, further aspects, embodiments, and features will become apparent by reference to the drawings and the following detailed description.

### SUMMARY

Described herein are various illustrative apparatus for an improved wearable medical device (WMD). Example apparatus may include a clothing and an electronic module of a WMD. The electronic module may be integrated with the clothing and be configured to facilitate operation of the clothing as WMD.

The present disclosure also describes a method of manufacturing an improved a wearable medical device (WMD). The method may include disposing one or more electronic modules within a clothing. The one or more electronic modules may have electronic components configured to facilitate operation of the clothing as a WMD. Additionally, the method may include integrating the one or more electronic modules with the clothing to facilitate accommodation of the electronic components.

The foregoing summary is illustrative only and not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

In the drawings:

FIG. 1 illustrates a wearable medical device (WMD), in accordance with various embodiments;

FIG. 2, illustrates a WMD having an integrated number of modules, in accordance with some embodiments;

FIG. 3 illustrates a WMD worn by a person, in accordance with one or more embodiments of the present disclosure;

FIG. 4 illustrates a WMD, in accordance with some alternate embodiments; and

FIG. 5 is a block diagram illustrating components of a defibrillator device, which may be used with various embodiments.

### DETAILED DESCRIPTION

The following description sets forth various examples along with specific details to provide a thorough understanding of claimed subject matter. It will be understood by those skilled in the art after review and understanding of the present disclosure, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

This disclosure is drawn, inter alia, to apparatus and systems related to a providing an improved wearable medical device (WMD) without the use of a separate carry bag for an electronic module.

Wearable medical devices (WMD) may be used to facilitate monitoring and treatment of various medical conditions of a person. To help facilitate monitoring and treatment of medical conditions of a person, a WMD may be formed as part of a garment or a wearable apparel. For example, the WMD may be formed as part of an apparel that may be worn by a person to help facilitate monitoring and treatment of potential issues with the person's heart (e.g., Arrhythmia, where the person may utilize a defibrillator). An example of a WMD configured to facilitate monitoring and treatment of a person's heart may be a WMD such as, but not limited to, a wearable cardioverter defibrillator (WCD).

A WCD, which may help facilitate prevention of Sudden Cardiac Death (SCD), may be formed as part of an apparel that the person may wear (WCD apparel). A WCD apparel may include multiple electrodes and one or more electronic modules. The multiple electrodes may be disposed within the WCD apparel in locations proximate to the person's heart (e.g., at various locations of the patient's torso). The one or more electronic modules may be integrated within the WCD apparel and be communicatively coupled to the multiple electrodes (e.g., electrically coupled). One or more of the multiple electrodes may be adhesive type electrodes. In other embodiments one or more of the multiple electrodes may be dry electrodes attached to or integrated in the WCD apparel. In some embodiments, one or more of the multiple electrodes may be textile electrodes attached to or integrated in WCD apparel. If the person experiences a heart condition requiring treatment, the WCD apparel (e.g., the one or more electrodes and the electronic module) may facilitate detection of the heart related issue, and responsive to the detected heart related issue, the WCD apparel may facilitate treatment of the person's heart via the one or more electrodes (e.g., defibrillate the person's heart).

Before turning to the figures, a non-limiting example configurations and utilization of the various embodiments of the present disclosure is described. In the non-limiting example, a wearable medical device (WMD) may be utilized to facilitate monitoring and treatment of a person. An example of a WMD to facilitate monitoring and treatment of a person may be a WMD to monitor and treat a person's heart such as, but not limited to, a wearable cardioverter defibrillator (WCD). In one example, the WCD may be in the form of a clothing such as, but not limited to, a vest (WCD vest).

A WCD vest may include one or more electrodes to facilitate an electric shock for the defibrillation process. Additionally, the WCD vest may include one or more electronic modules having many of the electronic components to facilitate monitoring and/or treatment of the heart. The one or more electronic modules and the one or more electrodes may facilitate reading of the activities of the heart and the administration of the treatment of the heart (e.g., an electric shock for defibrillation, cardioversion and/or pacing). Accordingly, the one or more electrodes may be disposed on the inside surface of the WCD vest proximate to the person's heart and/or close to or on the skin of the person. In an enhancement, in some embodiments additional adhesive electrodes not disposed in the WCD vest may be attached to the patient. In accordance with the present disclosure, the one or more electronic modules may be disposed within and integrated with the WCD vest resulting in a self-contained WCD vest without the one or more electronic modules being a separate component of the WCD vest.

The one or more electronic modules may comprise of various electronic components to facilitate operation of the WCD. For example, the one or more electronic modules may include a power supply such as, but not limited to, a battery to provide a defibrillator electrical shock to the person via the one or more electrodes. Along with the battery, the one or more electronic modules may include one or more capacitors as part of a discharge circuit for the shock. Additionally, the one or more electronic modules may include a user interface such as, but not limited to, a physical button (e.g., response buttons), graphical user interface (e.g., display, interactive and non-interactive), audible interface (e.g., indication sounds), etc. The operation and coordination of the electronic components may be facilitated by a processor included in the one or more electronic modules being communicatively coupled to the various electronic components to facilitate operation of the WCD. It should be appreciated after review of this disclosure that the above example components are just a few examples, and accordingly, an electronic component of a WCD may include a wide variety of electronic components to facilitate operation of the WCD. Additionally, some of details of the one or more electronic modules of a WCD will be described below.

Continuing with the non-limiting example of a WCD in the form of a vest, the example WCD vest may have one or more electronic modules integrated with the clothing (i.e., with the vest), in accordance with the present disclosure. In one example, the one or more electronic modules (i.e., the electronic components) may be in a form factor configured to be included in the vest such as, but not limited to, one or more electronic modules in the form factor of a smart tablet type device. The electronic module in the smart tablet type device form factor may be integrated with the vest by being contained in a sleeve or a pocket of the vest.

In another example, the one or more electronic modules may be included in a clothing configured to go around a person's body such as, but not limited to, a belt. In some embodiments, one or more of the electrodes are integrated into the belt. In some embodiments, one or more of the electrodes are adhesive type electrodes communicatively coupled to one or more of the electronic modules included in the belt. In still other embodiments, the electronic module belt may be worn around the body of the person along with the vest, and together, the belt and the vest may facilitate operation of the WCD (i.e., monitoring and defibrillation of a person's heart). The belt, itself, may be integrated with the vest. For example, the belt may be configured to be attached to the vest by some form of attachment mechanism such as, but not limited to, sewing, hook and loop fastener, zipper fastener, buttons, hooks and eye, snaps/press studs, etc.

In yet another example, the one or more electronic modules may not be in a form factor of a single unit, but instead, the various electronic components of the electronic module may be separate individual electronic components. The individual electronic components may be integrated with the vest and/or belt by being distributed within the vest and/or belt (e.g., inside a lining of the vest and/or belt) with the user interface being disposed on a surface of the vest and/or belt to facilitate interaction by a person. In some embodiments, the size and weight of the electronic components are distributed on the vest and/or belt to improve comfort, discreteness, wearability and/or usability. For example, to distribute weight and to reduce individual form factor, the battery and one or more capacitors may be in separate electronic modules to increase comfort and reduce the size and thickness of the vest and/or belt. Such reduced size and thickness can enable the WCD to be completely covered by the patient's normal clothing and/or if the WCD or a portion of the WCD is visible outside of the patient's normal clothing it will appear as normal apparel. In some embodiments, multiple capacitors are used, with a first set of one or more capacitors used to provide charge for a first phase of a biphasic defibrillation shock and a second different set of one or more capacitors used to provide charge for the second phase of the biphasic defibrillation shock. As will be appreciated after review of this disclosure, the individual electronic components may all be communicatively coupled (e.g., electrical communication via printed circuit boards, vias, wiring, tracings, electrical printing, cables, conductive ink, etc.).

Continuing with the non-limiting example, a person, who uses the above example WCD, may simply wear the WCD (i.e., the clothing) having the electronic components to facilitate proper operation of the WCD (e.g., monitoring and treating potential issues with a person's heart). Since the electronic components may be integrated with the vest and/or belt, the person would not have a separate component to be minded or carried. Accordingly, the comfort and convenience of using the WCD may be improved. For example, the person may put on the WCD in the morning and go about their activities as if the person was just wearing a piece of clothing (e.g., wear it and forget it). In some example, the WCD may be configured to be charged (i.e., the power supply). Even this charging may be performed either while the person wears the WCD or the person may take the WCD off to charge resulting in increased convenience of using the WCD. Additionally, the person may interact with the WCD via an interface, which may be integrated with the clothing (e.g., integrated display on a surface of the clothing).

It should be appreciated after review of this disclosure that the above non-limiting examples facilitate integration of an electronic module of a WCD with a clothing of the WCD. This integration facilitates comfort and convenience of use of the WCD promoting increased use of the WCD. Additionally, the integration of the electronic module with the clothing of the WCD reduces utilization of a separate carry bag (e.g., a carry bag for the electronic module).

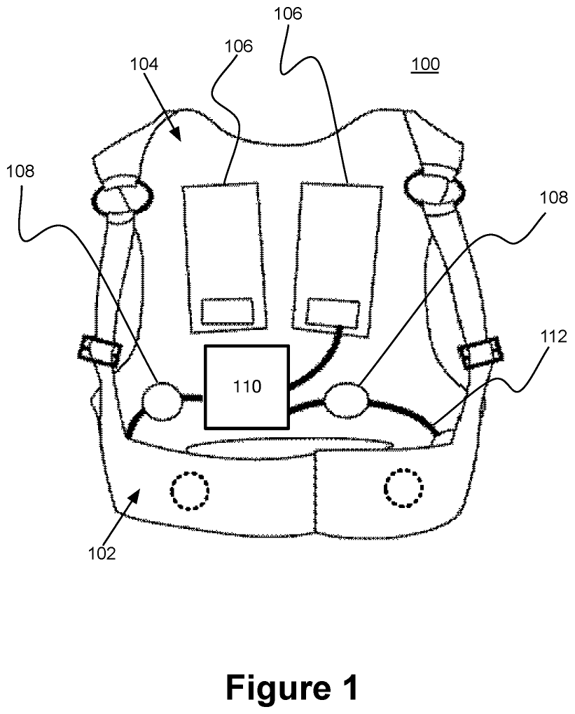

Turning now to FIG. 1, FIG. 1 illustrates a wearable medical device (WMD), in accordance with various embodiments. In FIG. 1, a WMD may be configured to facilitate monitoring and treatment of a person's heart such as, but not limited to, a wearable cardioverter defibrillator (WCD) 100. The WCD may be in the form of a clothing configured to be worn by a user such as, but not limited to, a vest type or a belt type clothing. Accordingly, the WCD 100 may have a front side 102 and a back side 104 forming the vest type WCD 100 as shown. Additionally, the WCD 100 may include one or more electrodes configured to defibrillate the person's heart, defibrillator electrodes 106 and one or more electrodes configured to detect and measure the person's electrocardiogram (ECG), ECG electrodes 108. It should be appreciated after review of this disclosure that the locations of the defibrillator electrodes 106 may be shown in various configurations such as, but not limited to, one front and one back, across a chest, across a back, etc. to facilitate defibrillation, and accordingly, the locations of the defibrillator electrodes 106 and/or the ECG electrodes 108 in FIG. 1 may be for illustrative purposes to show that there may be some electrodes to facilitate operation of the WCD 100. Continuing to refer FIG. 1, the WCD 100 may include an electronic module 110, in accordance with various embodiments. In the example shown in FIG. 1, the electronic module 110 may be integrated with the WCD 100 on the back side 104. The electronic module 110 may be communicatively coupled to the defibrillator electrodes 106 and to the ECG electrodes via one or more wires 112.

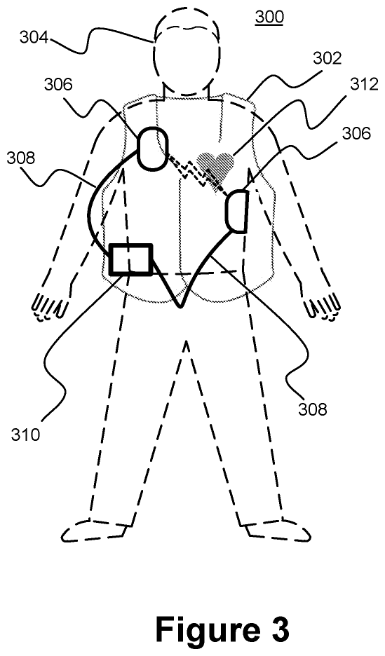

In FIG. 1, the WCD 100 may be considered to be a self-contained apparatus, in accordance with various embodiments. A person 304 (shown in FIG. 3) may wear the WCD 100 without consideration for a component that may be separate from the WCD (e.g., the electronic module 110) and may be carried by the person 204. Accordingly, the WCD 100 may be more comfortable and convenient to use (i.e., wear) for the person.

As will be described, the electronic module 110 may include various electronic components configured to facilitate operation of the WCD (i.e., monitor and defibrillate the person's heart). For example, the electronic module 110 may include a power supply such as, but not limited to, a battery to provide a charge for a defibrillator shock via the defibrillator electrodes 106.

It should be appreciated after review of this disclosure that the WCD 100 shown in FIG. 1 may be in the form of a vest. However, the WCD 100 may be in the form of a wide variety of clothing such as, but not limited to, a jacket, a t-shirt, a dress shirt, a belt, a blouse, a coat, and any combination thereof. Accordingly, the components of the WCD 100 may be integrated with a wide variety of wearable clothing and is fully contemplated within the scope of the claimed subject matter.

In FIG. 1, the various components of the WCD 100 may be shown integrated with the clothing on various surfaces. It should be appreciated after review of this disclosure that the method of attachment and integration of the various components of the WCD (e.g., defibrillator electrodes 106 and electronic module 110) may be facilitated by a variety of methodologies such as, but not limited to, sewing, hook and loop fastener, zipper fastener, buttons, hooks and eye, snap/press studs, use of adhesives, etc., and accordingly, the claimed subject matter is not limited in this respect.

It is contemplated within the scope of the disclosure that the material used for the clothing of the integrated WCD 100 may include a wide variety of material. Material such as, but not limited to, various fabrics, cotton type material, silk type material, synthetic type material, smart fabric material, moisture wicking material, animal-based type material, etc. may be utilized for the clothing of the integrated WCD 100. Accordingly, the claimed subject matter is not limited in this respect.

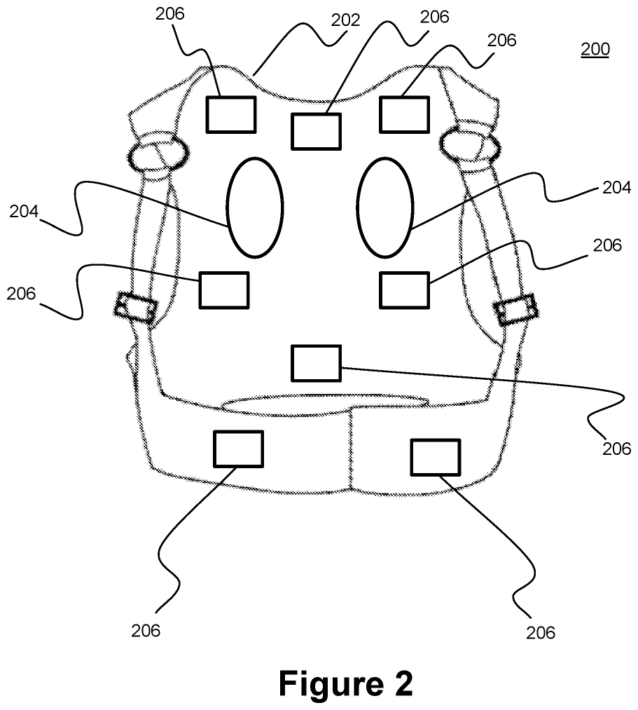

FIG. 2 illustrates a WMD having an integrated number of modules, in accordance with some embodiments. In FIG. 2, a WMD 200 may be configured to be a WCD 202 to be utilized by a person 304 (shown in FIG. 3). The WCD 202 may be in the form of a clothing having the shape of a vest. The WCD 202 may include at least one or more WCD components such as, but not limited to, two electrodes 204 (e.g., defibrillator electrodes as previously described) and one or more modules 206. The one or more modules 206 may be one or more electronic modules as previously described above. In accordance with the present disclosure, the WCD 202 may have the various components, two electrodes 204 and the one or more modules 206, included and integrated with the clothing resulting in a self-contained WCD 202.

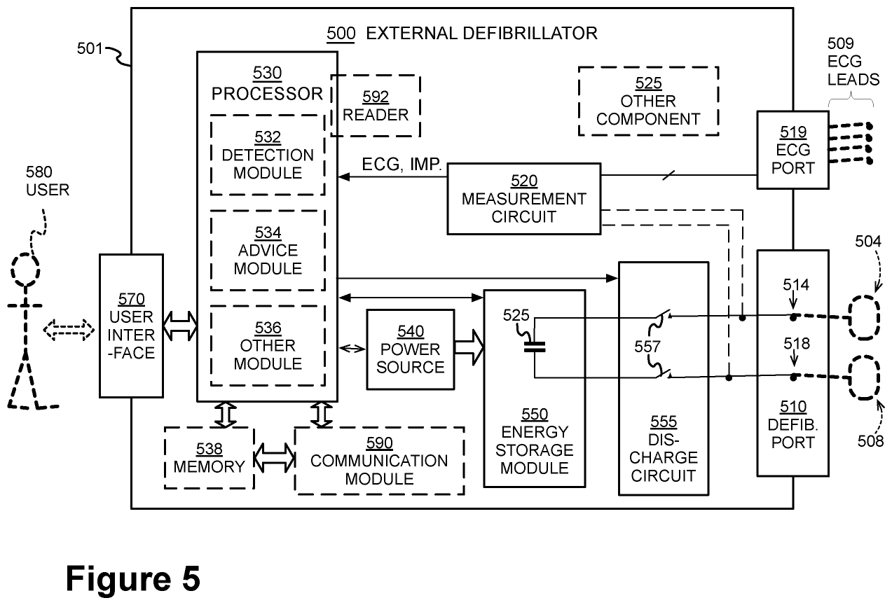

Shown in FIG. 2, the one to more modules 206 may be distributed within the WCD 202 as shown (i.e., dispersed throughout the vest). Each of the one or more modules 206 may represent one or more electronic components that may make up a module of a WCD. For example, the one or more modules 206 and with the two electrodes 204 may be communicatively coupled to facilitate the proper function of the WCD 202 (i.e., monitor and defibrillate a person's heart 312 (shown in FIG. 3). The one or more electronic modules 206 may comprise of one or more electronic components described below with respect to FIG. 5. Here again, it should be appreciated after review of this disclosure that even though the locations of the two electrodes 204 may be shown as being located on the back of the WCD 204 may be one front and one back, across a chest, across a back, etc. to facilitate defibrillation, and accordingly, the locations of the two electrodes 204 in FIG. 2 may be for illustrative purposes to show that there may be some electrodes to facilitate operation of the WCD 202.

Referring back to the non-limiting example scenario above, the person 304 may wear the WCD 202 without having a separate WCD component external to the clothing (e.g., the vest). The person 304 may simply wear the WCD (i.e., the vest) and have the WCD 202 function appropriately as a WMD 200 (i.e., a medical device for the treatment of the heart 312 of the person 304). As a result the use of the WMD 200 may be more comfortable, discrete, and convenient for the person 304.

In one example, the one or more modules 206 and the two electrodes 204 may be communicatively coupled via wires sewn into the fabric of the clothing. In some embodiments, the two electrodes 204 may be electrodes that both sense ECG and provide defibrillation and/or pacing therapy. In other embodiments, the two electrodes 204 are defibrillator electrodes and additional ECG electrodes are used to sense the patient's ECG signals. In another example, the one or more modules 206 and the two electrodes 204 may be communicatively coupled wirelessly utilizing wireless protocols such as but not limited to, Bluetooth® type, IEEE 802 based, mesh local area network (LAN) type (e.g., ZigBee, Bluetooth Low Energy, Z-Wave, 6LoWPAN, Thread, etc.), and any combination thereof. Alternatively, the one or more modules 206 may be communicatively coupled wirelessly, while the two electrodes 204 may be communicatively coupled via wires. For example, in the case of the two electrodes 204 being used for both therapy and ECG sensing, the wires can be used to deliver charge for therapy, while the wireless link can be used to communicate ECG signals, thereby protecting the ECG sensors from potential damage when therapy is delivered to the patient.

In yet another example, the material of the WCD 202 clothing may be made of smart textiles having integrated conductive textiles to facilitate communicatively coupling the one or more modules 206 such as, but not limited to, wireless body sensor networks based on metamaterial textiles. The clothing may include one or more sensors with the metamaterial textiles forming a number of metamaterial structures integrated into the clothing such as, for example, the metamaterial structures described in Tian, X., Lee, P. M., Tan, Y. J. et al. Wireless body sensor networks based on metamaterial textiles. Nat Electron 2, 243-251 (2019). One of the one or more sensors may be wirelessly coupled to one of the one or more modules 206 (e.g., electronic module) via the number of metamaterial structures. For example, the one or more sensors may include one or more ECG type sensors with the ECG type sensors being textile based electrodes integrated in the clothing.

It should be appreciated in view of this disclosure that it is within the scope of the disclosure that the one or more modules 206 and the two electrodes 204 may be communicatively coupled via any combination of the above described methodologies.

In FIG. 2, because the one or more modules 206 may be distributed in the WCD 202 (i.e., in the clothing), the one or more modules 206 may have form factors that help to facilitate integration of the modules 206 into the WCD 202 while providing improved comfort for the person 304 to wear the WCD 202. For example, a form factor of the one or more modules 206 may have a weight distribution such as, but not limited to, weight per unit area equal to or less than 0.1 pound per square inch. Additionally, a form factor of the one or more modules 206 may have a thickness to facilitate integration with the material of the WCD 202 such as, but not limited to, each of the one or more modules 206 having a thickness of equal to or less than 1 inch. Alternatively, the thickness may be based, at least in part, on the thickness of the material of the WCD 202 (i.e., material of the clothing). For example, the length and width of the form factor may be a variety of dimensions to be integrated with the WCD 202 and may vary based, at least in part, on the size of the clothing (e.g., size of the person 304).

In some examples, the one or more modules 206 may be distributed based, at least in part, on each of the one or modules 206 dimensional, mechanical, or electrical properties. For example, the one or more modules 206 may include a power supply such as, but not limited to, a battery. The battery may be integrated into a location of the WCD 202 proximate to the middle of the back, which may help distribute the weight of the battery and provide a larger area to accommodate a larger battery (e.g., thin film battery). In some examples, the battery may be in a form of a flexible lithium polymer type battery, which may be thin and flexible enough to be integrated into and/or between the layers of the material of the clothing.

The one or more modules 206 may include a mechanical interface (e.g., button). The button may be integrated into a location of the WCD 202 proximate the front of the WCD 202 to facilitate easy access for the person 304 (e.g., proximate to the chest area or proximate to the front side 102 (shown in FIG. 1). Alternatively, or in addition to the mechanical interface, one of the one or more modules 206 may include an interface such as, but not limited to, a graphical user interface (GUI). The GUI may be integrated with the clothing by being on a surface to facilitate interaction with a person, either wearing the WCD or observer (e.g., first responder). In order to facilitate integration and comfort, the GUI may be in the form of a flexible display type GUI.

As described above, it should be appreciated after review of this disclosure that the one or more modules 206 may be integrated in a variety of manners with the clothing of the WCD 202 such as but not limited to, outer surface, inner surface, between layers of the material, interleaved within the material, a part of the panel of the clothing, and any combination thereof. Accordingly, the claimed subject matter is not limited in this respect.

In FIG. 2, the one or more modules 206 may include one or more electronic modules such as, but not limited to, various components described with respect to FIG. 5 below. As described in the present disclosure, since the one or more modules 206 may facilitate proper functioning of the WMD 200 as configured for its intended purpose (e.g., WCD), it is contemplated within the scope of the present disclosure that a wide variety of WMDs may have various integrated modules. For example, WMDs may include a variety of WMDs related to various health conditions such as, but not limited to, Alzheimer's, Peritoneal Dialysis, sweat related issues, etc. Accordingly, the claimed subject matter is not limited in this respect.

It should be appreciated after review of this disclosure that the WMD 200 shown in FIG. 2 is a simplistic illustration and does not show all of the various components, electronic and non-electronic, of the WCD 202. However, the claimed subject matter disclosed contemplates that there may be numerous components of a WMD that may be integrated into the WCD 202 (i.e., the clothing), and accordingly, the claimed subject matter is not limited in this respect.

FIG. 3 illustrates a WMD worn by a person, in accordance with one or more embodiments of the present disclosure. In FIG. 3, a WMD 300 may be configured to be a WCD 302 to be utilized by a person 304. The WCD 300 may be in the form of a clothing having the shape of a vest. The WCD 302 may include at least one or more WCD components such as, but not limited to, two electrodes 306 (e.g., defibrillator electrodes as previously described), one or more wires 308, and a module 310, the module 310 may include an electronic module. In accordance with the present disclosure, the WCD 302 may have the various components, two electrodes 306, the one or more wires 308, and the module 310, included and integrated with the clothing resulting in a self-contained WCD 302.

Referring back to the non-limiting example scenario above, the person 304 may wear the WCD 302 without having a separate WCD component external to the clothing (e.g., the vest). The person 304 may simply wear the WCD (i.e., the vest) and have the WCD 302 function appropriately as a WMD 300 (i.e., a medical device for the treatment of the heart 312 of the person 304). As a result, the use of the WMD 300 may be more comfortable, discrete, and convenient for the person 304.

As previously mentioned, it should be appreciated after review of this disclosure that the WMD 300 shown in FIG. 3 is a simplistic illustration and does not show all of the various components, electronic and non-electronic, of the WCD 302. However, the claimed subject matter disclosed contemplates that there may be numerous components of a WMD that may be integrated into the WCD 302 (i.e., the clothing), and accordingly, the claimed subject matter is not limited in this respect.

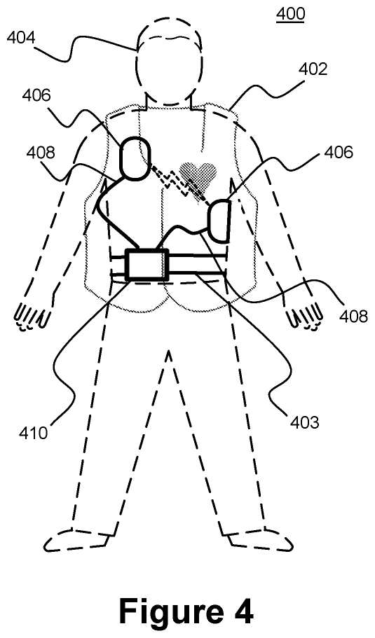

FIG. 4 illustrates a WMD, in accordance with some alternate embodiments. In FIG. 4, a WMD 400 may be configured to be a WCD 402 to be utilized by a person 404. Part of the WCD 402 may be in the form of a clothing having the shape of a vest. Another part of the WCD 402 may be in the form of a clothing in the shape of a belt 403. Similar to the WCD 302 shown in FIG. 3, the WCD 402 may include at least one or more WCD components such as, but not limited to, two electrodes 406 (e.g., defibrillator electrodes as previously described), one or more wires 408, and an module 410 (e.g., a module including an electronic module). Shown in FIG. 4, the electronic module 410 may be integrated into the belt 403.

In FIG. 4, the belt 403 having the module 410 may be worn around the body of the person 404. The belt 403 may be integrated with the WCD 402 by various attachment methodologies as previously described. In some embodiments, the one or more electronic modules are attached to or integrated into the belt, with one or more adhesive electrodes attached to the patient and communicatively coupled to the one or more electronic modules of the belt. In another example, the belt 403 may be worn with the vest (i.e., WCD 402) and may be considered to be an integrated WCD, in accordance various embodiments. For example, in some embodiments ECG and defibrillation electrodes are attached to or integrated into the vest and one or more electronics modules are attached to or integrated into the belt. In another example, the belt 403 may be attached to the vest (i.e., WCD 402) utilizing attachment methodologies such as, but not limited to, zipper fastener, hook and loop fastener, snaps/press studs, and any combination thereof. resulting in an integrated WCD, in accordance with various embodiments.

FIG. 5 is a block diagram illustrating components of a defibrillator device, which may be used with various embodiments. These components may be, for example, components of a WCD 100, 202, 302, and 402 (shown in FIGS. 1-4).

The defibrillator device 500 may be some of the above examples of an one or more modules for the WCD (e.g., electronic modules 110, 206, 310, and 410 shown in FIGS. 1-4) intended for use by a user 580 (e.g., a wearer or person 304 and 404 shown in FIGS. 3-4). The defibrillator device 500 may typically include a defibrillation port 510, such as a socket in housing 501. The defibrillation port 510 may include nodes 514 and 518. One or more electrodes 504 and 508, which may be plugged into the defibrillation port 510, so as to make electrical contact with nodes 514 and 518, respectively. It may also be possible that the electrodes 504 and 508 may be connected continuously to the defibrillation port 510, etc. Either way, the defibrillation port 510 may be used for guiding via the electrodes 504 and 508 to a person 504, and 304, and 404 shown in FIGS. 3 and 4 an electrical charge that may have been stored in the defibrillator device 500, as described herein.

The defibrillator device 500 may also have an ECG port 519 in the housing 501, for receiving ECG cables 509. The ECG cables 509 may facilitate sensing of an ECG signal (e.g., a 12-lead signal or from a different number of lead signals). Moreover, a defibrillator-monitor could have additional ports (not shown), and the other component 525 may be configured to filter the ECG signal (e.g., application of at least one filter to the signal to help facilitate removal of artifacts such as, but not limited to, chest compression due to chest compressions being delivered to the person).

The defibrillator 500 also may include a measurement circuit 520. The measurement circuit 520 may receive physiological signals from the ECG port 519, and also from other ports, if provided. The circuit 520 may render detected physiological signals and their corresponding information. The information may be in the form of data, or other signals, etc.

If the defibrillator 500 is configured as a WCD type device (shown in FIGS. 3 and 4), ECG port 519 may not be present. The measurement circuit 520 may obtain physiological signals through the nodes 514 and 518 instead, when the electrodes 504 and 508 are attached to the person (see FIGS. 2 and 3). In these cases, a person's ECG signal may be detected as a voltage difference between the electrodes 504 and 508. Additionally, the impedance between the electrodes 504 and 508 may be detected, among other things, whether the electrodes 504 and 508 have been inadvertently disconnected from the person.

The defibrillator 500 may also include a processor 530. The processor 530 may be implemented in a wide variety of manners for causing actions and operations to be performed. Some examples may include digital and/or analog processors such as microprocessors and digital-signal processors (DSPs), controllers such as microcontrollers, software running in a machine environment, programmable circuits such as Field Programmable Gate Arrays (FPGAs), Field-Programmable Analog Arrays (FPAAs), Programmable Logic Devices (PLDs), Application Specific Integrated Circuits (ASICs), and so on or any combination thereof.

The processor 530 may include a number of modules. One example module may be a detection module 532, which may detect outputs from the measurement circuit 520. The detection module 532 may include a VF detector. Accordingly, the person's detected ECG may be utilized to help determine whether the person is experiencing ventricular fibrillation (VF).

In another example module may be an advice module 534, which may provide advice based, at least in part, on outputs of detection module 532. The advice module 534 may include an algorithm such as, but not limited to, Shock Advisory Algorithm, implement decision rules, and so on. For example, the advice may be to shock, to not shock, to administer other forms of therapy, and so on. If the advice is to shock, some defibrillator examples may report the advice to the user, and prompt them to do it. In other examples, the defibrillator device may execute the advice by administering the shock. If the advice is to administer CPR, the defibrillator 500 may further issue prompts for administrating CPR, and so forth.

The processor 530 may include additional modules, such as module 536 for various other functions. Additionally, if other component 525 is provided, it may be operated in part by processor 530, etc.

In an example, the defibrillator device 500 may include a memory 538, which may work together with the processor 530. The memory 538 may be implemented in a wide variety of manners. For example, the memory 538 may be implemented such as, but not limited to, nonvolatile memories (NVM), read-only memories (ROM), random access memories (RAM), and so forth or any combination thereof. The memory 538 may can include programs for the processor 530, and so on. The programs may include operational programs execution by the processor 530 and may also include protocols and methodologies that decisions may be made by advice module 534. Additionally, the memory 538 may store various prompts for the user 580, etc. Moreover, the memory 538 may store a wide variety of information (i.e., data) such as, but not limited to information regarding the person.

The defibrillator 500 may also include a power source 540. In order to facilitate portability of defibrillator device 500, the power source 540 may include a battery type device. A battery type device may be implemented as a battery pack, which may be rechargeable or not be rechargeable. At times, a combination of rechargeable and non-rechargeable battery packs may be utilized. Examples of power source 540 may include AC power override, where AC power may be available, and so on. In some examples, the processor 530 may control the power source 540.

Additionally, the defibrillator device 500 may include an energy storage module 550. The energy storage module 550 may be configured to store some electrical energy (e.g., when preparing for sudden discharge to administer a shock). The energy storage module 550 may be charged from the power source 540 to an appropriate level of energy, as may be controlled by the processor 530. In some implementations, the energy storage module 550 may include one or more capacitors 552, and the like.

The defibrillator 500 may include a discharge circuit 555. The discharge circuit 555 may be controlled to facilitate discharging of the energy stored in energy storage module 550 to the nodes 514 and 518, and also to electrodes 504 and 508. The discharge circuit 555 may include one or more switches 557. The one or more switches 557 may be configured in a number of manners such as, but not limited to, an H-bridge, and so forth.

The defibrillator device 500 may further include a user interface 570 for the user 580. The user interface 570 may be implemented in a variety of manners. For example, the user interface 570 may include a display screen capable of displaying what is detected and measured, provide visual feedback to the user 580 for their resuscitation attempts, and so forth. The user interface 570 may also include an audio output such as, but not limited to, a speaker to issue audio prompts, etc. The user interface 570 may additionally include various control devices such as, but not limited to, pushbuttons, touch display, and so forth. Additionally, the discharge circuit 555 may be controlled by the processor 530 or directly by the user 580 via the user interface 570, and so forth.

Additionally, the defibrillator device 500 may include other components. For example, a communication module 590 may be provided for communicating with other machines and/or the electrodes. Such communication may be performed wirelessly, or via wire, or by infrared communication, and so forth. Accordingly, information may be communicated, such as person data, incident information, therapy attempted, CPR performance, ECG information, and so forth.

It should be appreciated after review of this disclosure that it is contemplated within the scope and spirit of the present disclosure that the claimed subject matter may include a wide variety of clips, materials, mechanical shapes, etc. Accordingly, the claimed subject matter is not limited in these respects.

In some portions of the description, illustrative implementations of the disclosure may have been described with reference to the elements of the components described with respect to FIGS. 1-5. However, the described embodiments are not limited to these depictions. More specifically, some elements/components depicted in FIGS. 1-5 may be omitted from some implementations detailed herein. Furthermore, other elements not depicted in FIGS. 1-5 may be used to implement example apparatuses detailed herein.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an implementation,” “one implementation,” “some implementations,” or “other implementations” may mean that a particular feature, structure, or characteristic described in connection with one or more implementations may be included in at least some implementations, but not necessarily in all implementations. The various appearances of “an implementation,” “one implementation,” or “some implementations” in the preceding description are not necessarily all referring to the same implementations.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter is not limited to the particular examples disclosed, but that such claimed subject matter also may include all implementations falling within the scope of the appended claims, and equivalents thereof.

1. A wearable cardioverter defibrillator (WCD) comprising:
a vest;
a belt;
an energy storage device;
an output circuit that is integrated into the belt or the vest;
a plurality of sensors that are attached to the vest, wherein the plurality of sensors are configured to monitor physiological conditions of a wearer of the vest and the belt;
defibrillator electrodes that are attached to the vest; and
a processor that is attached to the belt and is communicatively coupled to the plurality of sensors, the output circuit, and the defibrillator electrodes, wherein the processor is configured to:
receive data about the wearer from plurality of the sensors,
determine that the wearer is experiencing a cardiac event based on the data, and
cause the output circuit to deliver electrical charge from the energy storage device to the defibrillator electrodes to deliver a shock to the wearer in response to a determination that the wearer is experiencing the cardiac event.

2. The WCD of claim 1 wherein the energy storage device comprises:
a capacitor that is attached to the belt; and
a battery that is attached to the belt,
wherein the processor is further configured to cause the battery to charge the capacitor before causing the output circuit to deliver the shock.

3. (canceled)

4. (canceled)

5. (canceled)

6. The WCD of claim 2, wherein the capacitor, battery and processor are substantially uniformly distributed along the belt.

7. The WCD of claim 1, further comprising a user interface that is attached to the vest.

8. The WCD of claim 1, further comprising a user interface that is attached to the belt.

9. The WCD of claim 7, wherein the user interface comprises a flexible display.

10. The WCD of claim 1, further comprising:
a plurality of metamaterial structures integrated into the vest, wherein the plurality of sensors are configured to communicate electrocardiogram (ECG) signals sensed from the wearer to the processor via the plurality of metamaterial structures.

11. The WCD of claim 1, wherein the plurality of sensors include a plurality of electrocardiogram (ECG) sensors.

12. (canceled)

13. A method for making a wearable cardioverter defibrillator (WCD), the method comprising:
attaching a plurality of sensors in a vest, wherein the plurality of sensors are configured to monitor physiological conditions of a wearer of the vest:
attaching defibrillator electrodes to the vest;
attaching a processor to a belt;
attaching an energy storage device to the belt;
attaching an output circuit to the belt;
communicatively coupling the processor to the plurality of sensors, and the output circuit, and electrically coupling the output circuit to the defibrillator electrodes; and
configuring the processor to:
receive data about the wearer from plurality of the sensors,
determine that the wearer is experiencing a cardiac event based on the data, and
cause the output circuit to output electrical charge from the energy storage device to the defibrillator electrodes to deliver a shock the wearer in response to a determination the wearer is experiencing the cardiac event.

14. The method of claim 13, wherein attaching the energy storage device further comprises:
attaching a capacitor in the belt;
attaching a battery in the belt; and
further configuring the processor to cause the battery to charge the capacitor prior to causing the output circuit to deliver the shock.

15. (canceled)

16. (canceled)

17. (canceled)

18. (canceled)

19. The method of claim 13, further comprising:
communicatively coupling the plurality of sensors to the processor via a plurality of metamaterial structures in the vest, the plurality of sensors configured to communicate electrocardiogram (ECG) signals sensed from the wearer to the processor via the plurality of metamaterial structures.

20. (canceled)

21. The WCD of claim 1, wherein the belt is removably attached to the vest.

22. The method of claim 14, wherein the attaching of the capacitor, the attaching of the battery and the attaching of the processor are performed so that the capacitor, battery and processor are substantially uniformly distributed along the belt.

23. The method of claim 13 further comprising:
attaching a user interface in the vest.

24. The method of claim 23, wherein the user interface comprises a flexible display.

25. The method of claim 13 further comprising attaching a user interface in the belt.

26. The method of claim 13, wherein the plurality of sensors include a plurality of electrocardiogram (ECG) sensors.

27. The method of claim 13, further comprising:
removably attaching the belt to the vest.
